(19) United States
(12) Patent Application Publication
(10) Pub. No.: US 2010/0189712 A1
(43) Pub. Date: Jul. 29, 2010

(54) Preparation And Use Of Cell-Synthesized Threads

(75) Inventors: Nicolas L'Heureux, Corte Madera, CA (US); Todd N. McAllister, San Anselmo, CA (US)

Correspondence Address: FISH & RICHARDSON P.C., PO BOX 1022, MINNEAPOLIS, MN, 55440-1022, US

(73) Assignee: CYTOGRAFT TISSUE ENGINEERING, INC., Novato, CA (US)

(21) Appl. No.: 12/515,397

(22) PCT Filed: Nov. 19, 2007
(86) PCT No.: PCT/US07/85148

§ 371 Date: Oct. 28, 2009

Related U.S. Application Data
(60) Provisional application No. 60/859,539, filed on Nov. 17, 2006.

(51) Int. Cl. A61K 35/12; A61K 39/395; A61K 35/76; C12N 5/071; C12N 5/0735; C12N 5/078; C12N 5/074; C12M 1/24; C12N 5/079; A61K 35/28; A61K 35/30; A61K 35/34; A61K 35/36; A61P 31/00

(52) U.S. Cl. 424/130.1; 424/93.7; 424/93.6; 435/366; 435/371; 435/372; 435/368; 435/304.1

(57) ABSTRACT

The present technology provides for a cell-synthesized biological thread, processes for making a cell-synthesized thread, and an apparatus for carrying out a process used to engineer the cell-synthesized thread. The thread is produced from living cells in culture and can be grown around the outer surface of a cylindrical bioreactor. The tissue comprising the thread has mechanical and biological properties achieved by altering the climactic conditions, stresses and strains on the tissue and chemical composition that prove beneficial in the medical industry. This process results in a biological thread that has high mechanical strength, decreased immunogenic effect and decreased thrombogenic effect when combined with other tissue. It also results in a product which can be used to create more complex constructs that otherwise could not have been generated. The threads can be used to make sutures, patches, and tubes, for example.

### CLAIM OF PRIORITY

This application claims the benefit of priority under 35 U.S.C. §119(e) to U.S. provisional application Ser. No. 60/859,359, filed Nov. 17, 2006, which is incorporated herein by reference in its entirety.

### TECHNICAL FIELD

The present technology relates to the field of tissue engineering. More specifically, the technology relates to the technical field of threads exclusively engineered from cells in culture, and their subsequent use.

### BACKGROUND

Multicellular organisms are made up of tissues, that is, organized aggregates of specialized groups of cells of similar form and function. Tissue defects can arise from diverse medical conditions, including, for example, congenital malformations, traumatic injuries, infections, and oncologic resections. Successful repair of defective or damaged tissue depends in part on providing conditions that allow for appropriate cellular regeneration and that minimize the likelihood of infection and inflammation during the repair process. Because ineffective wound healing can result in serious complications, for example, chronic wound formation, venous ulcers or keloid scars, there is a continuing need for therapeutic strategies that promote tissue regeneration.

Tissue engineering emerged from a convergence of the fields of medicine and engineering. Tissue engineering includes techniques and methods for producing tissue outside the human body, such as in vitro. For hundreds of years, surgeons have dealt with the problem of repairing and replacing damaged tissue in living organisms. The most commonly used methods of implantation, transplantation and prosthetics provide only a rudimentary solution to the diverse number of possible problems and eventually meet their practical limitations. Traditionally, tissue engineering has attempted to solve these problems using the principles of cell biology in conjunction with the use of material science to produce complex medical devices. However, the use of artificial materials has caused drawbacks ranging from adverse immunogenic effects to complete rejection by the host organism. More recent approaches have focused on the belief that the development of completely biological materials will allow for greater success in developing therapeutic strategies aimed at the repair, replacement, maintenance and enhancement of tissue function. However, development of purely biological materials is not without its own obstacles.

It has been observed that during long-term culture in the presence of ascorbic compounds, adherent cells, such as fibroblasts, can lay down large amounts of extracellular matrix proteins on culture dishes. As a result, planar living tissues, comprised of the living cells and the extracellular matrix proteins produced by the cells, can be detached from the culture dishes in sheets. These living sheets have formed the basis for the development of a method, termed Sheet-Based Tissue Engineering (“SBTE”), which allows for the production of living and completely biological constructs that have high mechanical strengths without the need for artificial or other exogenous scaffolding. See, for example, L'Heureux et al., FASEB J., 12 (1), 47 (1998); L'Heureux et al., Nat. Med., 12 (3), 361 (2006); U.S. Pat. No. 7,112,218 and U.S. patent application Ser. No. 10/318,279, published as U.S. 2003-0138945, all of which are incorporated herein by reference. Such a method involves stacking sheets and then wait for them to adhere and strongly fuse together while in culture to produce thicker planar tissue, or rolling sheets to produce tubular tissues. See also, e.g., Michel et al., In Vitro Cell. Dev. Biol.-Anim., 35 (6), 318 (1999), and U.S. patent applications Ser. Nos. 10/522,010 and 10/198,628). In both cases, the sheet layers must be maintained in close apposition for an extended period of time, typically two months, to achieve fusion of the different layers. This period of time, referred to as the maturation period, involves regular media replenishment and maintenance of a controlled environment. The maturation period has been found to be essential to develop adequate mechanical strength and cohesion of the engineered tissues or organs. It would be greatly beneficial to eliminate or minimize this maturation period from both an economic and medical standpoint, since delays in production and availability of replacement tissues can adversely impact the health of patients awaiting treatment.

One solution to make tissue-engineered materials more rapidly available has been to use scaffolds of reconstituted extracellular matrix proteins such as collagen, elastin and fibronectin. These proteins can be cast into different shapes or can be made into fibers and assembled into three-dimensional constructs using processes known in the art, such as electrospinning Cells can then be added to the constructs to mimic the physiological function of a specific tissue. One of the key limitations to this approach is the inability to produce threads or tissues with adequate mechanical strengths. This is due in part to the fact that proteins are often denatured or chemically modified by the extraction process needed to obtain the more or less purified proteins prior to reassembly in engineered constructs. When reconstituted into a tissue in vitro, these modified proteins lack the microstructure of the original proteins or lack one or more required associated protein needed to recreate the microorganization of the extracellular matrix. In most cases, even an intact protein will fail to reassemble spontaneously into the complex organization observed in nature without the intervention of living cells. In addition to poor mechanical strength, the abnormal structure and organization of extracted proteins is recognized by the non-specific immune system of the host as a “damaged” tissue, which leads to rapid degeneration, loss of function and mechanical strength. If the proteins are of animal origin and used in humans, they can trigger recognition by the specific immune system and lead to rejection.

Hence there is a need to develop a material which contains the same microstructure and microorganization of tissues found in nature, provides adequate mechanical strengths for the proposed physical function, avoids adverse immunogenic responses, and will eliminate the need for a lengthy maturation period.

The discussion of the background to the technology herein is included to explain the context of the technology. This is not to be taken as an admission that any of the material referred to was published, known, or part of the common general knowledge as at the priority date of any of the claims.

Throughout the description and claims of the specification the word “comprise” and variations thereof, such as “comprising” and “comprises”, is not intended to exclude other additives, components, integers or steps.

### SUMMARY

The present technology discloses a cell-synthesized biological thread, the process for engineering a cell-synthesized thread and the bioreactor device which employs the process used to engineer the cell-synthesized thread. The thread is originally produced from living cells in culture which are grown around the outer surface of a cylindrical bioreactor. The tissue comprising the thread has mechanical and biological properties achieved by altering the climactic conditions, stresses and strains on the tissue and chemical composition that prove beneficial in the medical industry. This process results in a biological thread that has high mechanical strength, decreased immunogenic effect and decreased thrombogenic effect when combined with other tissue. It also results in a product which can be used to create more complex constructs that otherwise could not have been generated.

The present technology additionally includes a tissue engineered biocompatible thread composition comprising one or more biological cells and an extracellular matrix synthesized by the cells. The thread composition may be isolated from, e.g., a culturing apparatus prior to use.

The present technology also includes a process for making a tissue engineered biocompatible thread composition, the process comprising: (a) seeding a population of cells in a culture vessel; and (b) culturing the cells under conditions that allow the formation of a tissue sheet comprised of cells and extracellular matrix synthesized by the cells in contact with the surface of the culture vessel; and (c) cutting the sheet into one or more strips thereby forming a biocompatible thread composition.

The present technology further comprises a method of making a tissue engineered biocompatible thread composition, the method comprising: (a) seeding a population of adherent cells on the surface of a cylinder contained within a culture vessel, wherein the surface of the cylinder comprises a plurality of circumferential grooves; and (b) culturing the cells under conditions that allow the formation of a tissue sheet comprised of cells and extracellular matrix synthesized by the cells in contact the grooves; and (c) removing the tissue sheet from the grooves, thereby forming a biocompatible thread composition.

A tissue engineered biocompatible thread composition made by the methods described herein.

Use of the biocompatible thread composition as described herein in the preparation of a medicament.

A method of treatment comprising: (a) identifying a mammalian subject as having a recipient organ or tissue in need of repair; and (b) placing the biocompatible thread composition described herein, or the tissue engineering construct described herein, in or on the organ or tissue.

An article of manufacture comprising: a measured amount of a tissue engineered biocompatible thread composition and one or more of packaging material, or a package insert comprising instructions for use, and a sterile container.

A bioreactor for use in growing tissue comprising: a cylindrical support having an external surface capable of supporting cellular growth; wherein the cylindrical support is enclosed in a chamber configured to provide conditions for cellular growth.

### BRIEF DESCRIPTION OF THE DRAWINGS

FIG. 1a illustrates a plan view, from the top, of one embodiment of the present technology, showing a cutting pattern to create one or more cell-synthesized threads from a tissue sheet grown in a planar tissue culture dish.

FIG. 1b illustrates a plan view, from the top, of another embodiment of the present technology, showing a cutting pattern to create one or more cell-synthesized thread from a tissue sheet grown in a planar tissue culture dish.

FIG. 2a illustrates a perspective view of a part of an apparatus for growing tissued engineered threads according to another embodiment of the present technology, and showing a cutting pattern to create a cell-synthesized thread, such as a seamless thread, from a tissue sheet grown around the outer surface of a cylinder.

FIG. 2b illustrates a cross-sectional view of grooves on the outer surface of a cylinder, as may be part of another embodiment of an apparatus for the production of cell-synthesized threads.

FIG. 3a illustrates a plan view of another embodiment of the present technology, using cell-synthesized threads to create a simple woven patch.

FIGS. 3b and 3c are photographs of a, respectively, living and dried woven patch made from human cell-synthesized threads (12×13 threads).

FIG. 3d is a micrograph depicting the intersection of threads in a dried woven patch made from cell-synthesized threads.

FIG. 4 shows a micrograph of various threads compared to a 4-0 prolene suture (about 180 μm) in diameter, shown in blue (third strand down from top). The other threads were made by methods described herein.

### DETAILED DESCRIPTION

Disclosed herein are apparatus, materials and methods relating to the production and use of cell-synthesized biocompatible thread compositions for the repair of damaged or defective organs or tissues, as well as the biological threads themselves. The biological threads can be created using techniques of tissue engineering. The threads have very high mechanical strengths and additionally can be assembled into even stronger and more complex three-dimensional constructs for use as tissue or organ replacement, as drug delivery systems or wound healing devices. The threads can also be used as an alternate to normal suture material for applications such as wound closure. In certain instances, load carrying proteins, such as collagen or elastin, are assembled by the cells in a near-natural physiological organization and can be produced by human cells. The threads are also biocompatible and, optionally, biodegradable.

During long-term culture in the presence of ascorbic compounds, adherent cells, such as fibroblasts, can lay down large amounts of extracellular matrix proteins on culture dishes, thereby creating sheets. As a result, planar living tissues, comprised of the living cells and the extracellular matrix proteins produced by the cells, can be detached from the culture dishes in sheets. These living sheets allow for the production of living and completely biological constructs that have high mechanical strengths without the need for artificial or other exogenous scaffolding. Exemplary methods and apparatus for creating such sheets are described in L'Heureux, et al., FASEB J., 12 (1), 47, (1998); L'Heureux, et al., Nat. Med., 12 (3), 361, (2006); U.S. Pat. No. 7,112,218 and U.S. patent application Ser. No. 10/318,279, all of which incorporated herein by reference.

In one embodiment of the technology described herein, “cell-synthesized threads” are created from a tissue sheet by cutting it into thin strips. These strips can be very narrow (filament-like) or wider (ribbon-like). Cohesive and completely biological living tissues with instantly high mechanical strength and complex geometry can be produced from the living cell-synthesized threads by methods known in the textile arts such as, but not limited to, weaving, knitting, knotting, braiding, stitching, crocheting, pressing or any combination thereof.

Other methods of producing such threads, as further described herein, can include growing the cell sheets in narrow channels formed on a cell culture surface. This surface can have a multitude of shapes, but is typically convex, and in one embodiment would be formed on the outer surface of a cylinder such that tissue contraction would tighten the thread onto the cylinder rather than causing the spontaneous detachment of the tissue as would occur for growth on its interior.

Still other methods include producing a tissue sheet, cutting a ribbon or band of the sheet, and twisting, such as winding, the ribbon about itself to form a twisted or braided thread. Multiple, such as two or more, ribbons can be intertwined, e.g., as a double, triple, etc., helix about one another to form a multi-stranded thread.

The threads can be produced from autologous or non-autologous human cells (allogeneic), from animal cells (xenogeneic), from genetically modified cells (human or animal) or any combination thereof. While fibroblast is the preferred cell type for thread production, threads can be produced using other cell types such as, but not limited to, myofibroblasts, myocytes or their precursors, smooth muscle cells or their precursors, mesenchymal stem cells, endothelial cells or their precursors, mesothelial cells, fat-derived stem cells, keratinocytes, macrophages, neurons or their precursors, glial cells or their precursors, islet cells or their precursors, hepatocytes or their precursors, osteoblasts or their precursors, or a combination of cell types. Certain stem cells may have key advantages in terms of immunogenicity, however, the concept of thread-based tissue engineering as described herein is not limited to particular cell type(s). In some embodiments the cells are not smooth muscle cells and the cell culture medium does not include smooth muscle cells.

Threads formed by methods described herein are completely biological but mechanically sound. The threads can be formed completely from human cells and even autologous to avoid immune reactions. The threads can also be living to provide desirable biological effect or advantageous healing. The methods and threads described herein have several advantages over tissue sheets. Threads as described herein eliminate the maturation period otherwise necessary to create a cohesive and mechanically sound tissue. The threads facilitate the production of complex geometries because of the versatility of the various knitting and weaving technologies. Threads permit a much better control of the mechanical properties of a tissue construct formed from them since, using knitting and weaving methods known in the art, it is possible to create tissues with controlled local geometrical variations such as, but not limited to, thickness, density and thread orientation which, in turn, lead to variations in local mechanical properties such as mechanical strength, elasticity and porosity. Threads can create cohesive tissue of essentially unlimited thickness because, unlike sheet-based tissue engineered constructs, the cohesiveness does not rely on sheet fusion, which is limited by the diffusion of gas and nutrients through the layers of sheets. Threads allow the production of seamless constructs of essentially unlimited lengths or width unlike sheet-based tissue engineered constructs, which are limited by the size of the culture containers. Such production can be achieved by combining cell-synthesized threads from multiple culture containers into one continuous construct by using methods well known in the arts of textile production such as, but not limited to, tying, gluing or crimping multiple threads in series. Taken together, these characteristics provide a significant advantage by allowing the production of vascularized bulky organs. Again, taking advantage of the various weaving or knitting techniques, bulky organs can be assembled with a network of pores or channels that can be connected to the body's vasculature.

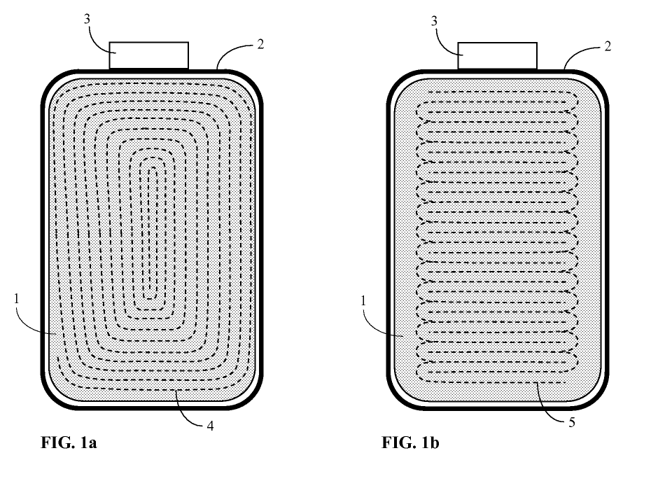

A thread can be formed from a tissue sheet by cutting the sheet into multiple straight strips or into a long single strip by cutting, for example, a generally spiraled pattern or a variety of sinuous patterns. FIGS. 1A and 1B show two embodiments of creating a thread from a tissue sheet grown in a culture dish 2, such as one having a planar surface, and having a cap 3. Referring first to FIG. 1a, a top view of one embodiment of the present technology is illustrated. Sheet 1 of living tissue can be grown in standard planar culture dish 2 with cap 3. Sheet 1 can be cut in a variety of shapes and lengths including multiple straight strips, but preferably in one continuous longer strip. This longer strip, which spirals around dish 2, can be made, for example, by cutting along dashed-line 4. Other intermediate length strips can also be cut from sheet 1. Complex patterns of cuts can also be made to form various networks of strips which include, but are not limited to branched and meshed strips.

Referring now to FIG. 1b, another top view of one embodiment of the present technology is illustrated. In FIG. 1b, sheet 1 grown in a planar culture dish 2 with cap 3 is also cut into one continuous longer strip. This longer strip, which weaves back and forth sinusoidally across dish 2, can be made for example, by cutting along dashed-line 5. Other various permutations of these patterns can also be envisioned.

The cutting can be achieved by means of a mechanical device such as a blade, a cutting wheel or a needle. It could also be performed using light, sound, thermal or other forms of energy. In order to achieve greater precision, the cutting can be automated and computer controlled or assisted. The cutting can be performed at any stage of the tissue growth and may be done before, during or after detachment of sheet 1 from dish 2. In the preferred embodiment, sheet 1 will be maintained alive prior to detachment to allow for further tissue contraction, cell proliferation and other remodeling changes. If sheet 1 is cut while attached to dish 2, the detachment of the cell-synthesized thread can be achieved mechanically, enzymatically, chemically, thermodynamically or using other methods known in the art. In one embodiment, the cutting results in perforated lines that allows the detachment of the thread by pulling the leading edge of the cutting pattern. This method can facilitate the handling of the thread after and during cutting by avoiding a free-floating state. Threads can also be produced without cutting if sheet 1 is originally grown in the shape of a ribbon or strip.

After detachment, the cell-synthesized threads can be used directly or maintained alive to allow tissue contraction or other biological changes to occur. The cell-synthesized threads can be kept unloaded or mechanically conditioned under stress in one or multiple directions using compressive, shear, tensile, isometric, isotonic or modulated forces produced from a variety of sources. Inducing fluid shear stress on the threads may be particularly important in producing a useful and beneficial product. The living thread can be stored on a spool or any other storage system known in the art to control thread length, apply mechanical stresses and strains or establish other environmental conditions while maintaining viability of the thread. The resulting thread can also be cryogenically preserved and stored frozen using methods known in the art to maintain partial viability.

At any time prior to, during or after detaching a cell-synthesized thread from the culture substrate, or prior to cutting a tissue sheet to create a cell-synthesized thread, the cell-synthesized threads or sheet can be devitalized to create a decellularized sheet or thread. The devitalization can be complete or partial. It can be achieved by any methods known in the art including but not limited to drying, heating, cooling, freezing, adding chemicals, such as acids, enzymes, salts, toxins or solvents, or applying various forms of energy, including, but not limited to ultrasound, radiation, mechanical forces and osmotic pressure. In the preferred embodiment, the thread is twisted while it is air-dried to create a flexible, homogenous and more elastic thread that can be stored at room temperature. The devitalization process may have secondary effects and be performed for ulterior purposes, such as increasing mechanical strength, reducing immunogenic or thrombogenic properties or improving the chances of implantation success.

In addition or alternatively, the cell-synthesized thread or the sheet can be treated with more or less powerful cross-linking agents including aldehydes, which can be used to achieve a complete or partial devitalization, to reduce immunogenic effects, to retard biodegradation, to modify mechanical properties or simply to attach chemical or biological compounds to the thread or sheet directly. This, in effect, forms a composite thread which can be altered in any or all of the ways previously described.

A composite thread, as described elsewhere herein, can also be decellularized and/or cross-linked under conditions such as the foregoing. The living thread can be devitalized at any of the production steps described herein. During or after the devitalization process, the living thread can be kept unloaded or mechanically conditioned, in any or in multiple directions, by compressive, magnetic, isometric, isotonic, modulated or other types or regiments of forces including fluid shear stress. The decellularized thread can be stored in various liquids, dried or frozen.

At any time during the production of the cell-synthesized thread, the living or decellularized thread, or the sheet used to create the thread, the product can be coated with various agents, including, but not limited to exogenous extracellular matrix proteins, natural deoxyribonucleic acid (“DNA”), recombinant DNA, natural ribonucleic acid (“RNA”), recombinant RNA, viral agents, transfection agents, growth factors, antiproliferation agents, antibodies, antibiotics or antiseptics or any fragment or combination of these compounds.

Before, after or at any time during a process of making a thread, the living or decellularized thread, or the sheet used to create the cell-synthesized thread, can be seeded with new cells. The cells can be of the same or of a different type than the ones used for the thread production, or a combination of cell populations. These cells can be non-autologous human cells (allogeneic), animal cells, genetically modified cells (human or animal) or any combination thereof.

Furthermore, the sheets can be cultured in the presence of exogenous elements that will become part of the sheet and, ultimately, the resulting thread. These elements include the list above but can also be macroscopic structures such as protein aggregates, natural or synthetic fibers such as synthetic sutures or cat-gut, and mineral, plastic or metallic devises. These could include needles, anastomotic devices, drug delivery devices or, magnetic or electronic devices, such as radio frequency ID tags. Besides identification, these devices can serve to facilitate or enhance further manipulation, storage, surgical use or healing. A

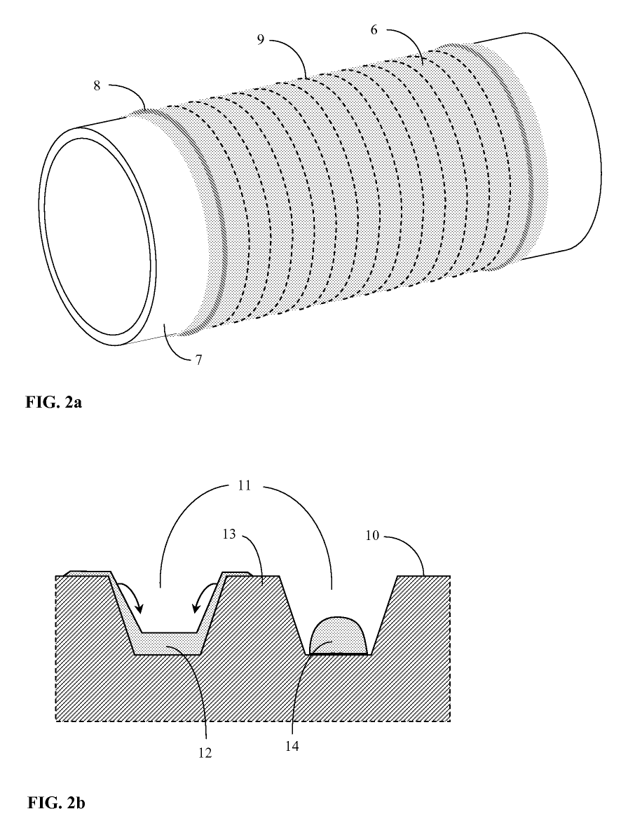

Referring now to FIG. 2a, a perspective view of a part of an embodiment of an apparatus use with the present technology is illustrated. Such an apparatus is often called a bioreactor. This type of culture system can be used to facilitate the production of a completely biological and seamless coiled living tissue 6. While using culture system, the culture substrate is an outer surface 7 of a generally cylindrical object. While a cylinder is the preferred shape for system, a tube or rod with faceted outer surface 7 having any number of sides, for example that would result in a triangular, square, rectangular, pentangular, etc. cross-section, can also be envisioned. In the following description the term “cylinder” and “cylindrical” is meant to include any of these possible shapes. Typically the cylinder is solid or capped so that it does not have an interior surface accessible to cells during the culturing process. The cylinder itself can be constructed of plastics, polymers such as polystyrene, metals or any material that will allow cell attachment. Outer surface 7 can also be treated with a number of chemicals and compounds that would modify its surface properties, and make it more adherent to cells. The cylinder can have a diameter that facilitates sheet formation on its exterior. In some embodiments, the cylinder has a diameter of 1 cm. In other embodiments, the cylinder has a diameter between 0.5 and 25 cm. In other embodiments the cylinder diameter is from 1 to 10 cm, and in still other embodiments, the cylinder has a diameter from 2-8 cm. Such exemplary diameters are non-limiting. It would be understood that the diameter is chosen appropriate to the length and thickness of thread that is desired.

The cylinder is typically inside of a closed container (not shown in FIG. 2a) such as a chamber to provide adequate sterility, climatic conditions or other culturing conditions appropriate for cell growth and well known in the art. In one embodiment of the present technology, the container could be equipped with a filter to provide adequate gas exchanges. In another embodiment of the present technology, the container could be placed in an incubator which could also provide beneficial or desirable conditions. The cylinder can be partially or completely bathed in culture media. If partially covered in media, the cylinder would typically undergo a form of physical displacement, possibly including rotation, to avoid drying of the uncovered surface. Alternatively, the media could be circulated to ensure adequate coverage of outer surface 7. The cylinder can be cultured vertically, horizontally or in any imaginable direction. Cells could also be seeded onto the cylinder any number of times by bathing the cylinder in a suspension of adherent cells, independent of media circulation or cylinder displacement. The culture media used to bath the cylinder would preferably be replenished regularly, typically trice weekly, to allow for adequate cell proliferation and extracellular matrix production. In some embodiments, the media would contain ascorbate or ascorbic derivatives, and animal serum or other sources of growth factors.

The culturing system described herein differs significantly from other methods known in the art, including the use of a “roller bottle” where cells are grown on the inner surface of a cylindrical object such as a bottle. A significantly thick and mechanically sound tissue cannot be produced from the previously used methods because the tissue rapidly detaches from the interior, such as a concave surface, of the culturing system. This is also a common problem in the production of tissue sheets grown in planar culture dishes. The present technology solves that problem by constraining the cells to grow on to and around a convex surface. When the cells are cultured around the outside of a cylinder, the lateral forces generated by the cells result in a force that constricts the tissue around the cylinder, thereby avoiding tissue detachment. Using culture system, tubes and seamless threads of living tissue can be formed and cultured for extended periods of time, allowing them to become thick and acquire the mechanical properties required for further processing into threads as further described herein.

In another embodiment, one or more control rods would be added to the cylinder. Control rods are structures present during the culture of a sheet and become embedded into the sheet. See for example U.S. patent application Ser. No. 10/318,279. The control rods provide anchoring to avoid contraction and early detachment of the sheet, as well as a way to manipulate the sheet more easily after detachment. As shown in FIG. 2a, the apparatus includes control rods using two metal rings 8 on opposing ends of tissue 6. These rings would get embedded during the development of the cylindrical sheet of living tissue and prevent or diminish longitudinal contraction of the tissue.

Other methods and techniques can also be used to avoid longitudinal contraction, including, but not limited to, addition of multiple perforations and/or protrusions to the culture surface, the use of porous material for outer surface 7, surface treatments to increase adhesion, air pressure, an elastic sleeve or mesh, clamps or ties that are included at different stages of sheet production. As is the case with planar sheets, other exogenous elements can also be present during the growth phase of the living cylindrical tissue, which would become embedded into tissue 6. These could include needles, anastomotic devices, drug delivery devices or, magnetic or electronic devices, such as radio frequency ID tags. Besides identification, these devices could serve to facilitate or enhance further manipulation, storage, surgical use or healing.

The culturing usually takes between four and sixteen weeks before the cells have laid down enough extracellular matrix to compose a strong cohesive living tissue but can be extended further to get a thicker tissue. To create a cell-synthesized thread from a cylindrical living tissue, a blade can be placed on outer surface 7, roughly perpendicular to longitudinal axis and normal to outer surface 7, to cut the pattern shown by dashed line 9. Tissue 6 is cut as the cylinder is simultaneously translated along and rotated around its longitudinal axis. Similar to the cutting of a sheet previously described, various cutting tools and methods can be applied. This cutting allows for the production of a continuous living thread using the entirety of the cylindrical tissue. As described previously, intermediate length strips and various shaped strips can also be cut. Complex patterns of cuts that involve, but are not limited to, branching and meshing to form networks of strips are also envisioned. Various thread widths can be produced by controlling the displacement of the cylinder. The width can be constant or varied depending on the particular application required. It should be understood that all the embodiments described elsewhere herein in the growing, cutting, modification, manipulation and detachment of a tissue sheet such as devitalization, cross-linking and seeding also apply to the production of a cell-synthesized thread.

Referring now to FIG. 2b, a cross-sectional view of an embodiment of a substrate according to the present technology is illustrated. Culture surface 10 can be grooved to form a long spiral around the cylinder. Grooves 11 can be made using any of the methods known in the art and can have various depths, widths, cross sections and geometries, including but not limited to curved such as semi-circular, straight such as square or rectangular, or polygonal such as triangular, or wedge shaped. The cells can be seeded in grooves 11 or tissue 12 could be grown in grooves 11 as shown on the left side of FIG. 2b. Cells and tissues growing on the sides of grooves 11 or on the ridge 13 between grooves 11 will be, for the most part, brought into the living thread as the tissue contracts toward the center of grooves 11 (shown by arrows).

After an extended culture period in conditions appropriate for sheet production, shown on the right side of FIG. 2b, the living thread 14 would form in grooves 11. Thin layers of cells can remain between the living thread spirals and will be torn when the living thread is removed from the cylinder. Alternatively, ridge 13 between grooves 11 can be contacted with a surface or tool to actively remove, cut, or damage developing tissue around ridge 13. Alternatively, ridge 13 can be treated to inhibit cell adhesion. A spiral of cultured cells around a cylinder can also be formed using one of many micropatterning methods known in the art, such as lithography, laser treatment or photo-activation, to create a surface-treated spiral to allow cell adhesion and proliferation on an otherwise non permissive material. Alternatively, a surfaced-treated spiral to inhibit cell adhesion and proliferation can be created on an otherwise permissive material. In all possible embodiments, an anchoring method such as the use of control rods could be used to avoid contraction or detachment from the extremities of the spiral. Alternatively, the spiral could begin and terminate in a groove or culture permissive region that forms a complete ring around the cylinder. All the grooving and micropatterning strategies described elsewhere herein can also be used to create cell-synthesized threads from conventional planar culture surfaces.

The cell-synthesized threads can be used to produce various two and three-dimensional constructs. These constructs can undergo the same treatments and procedures previously described for individual threads. For example, the constructs can be maintained alive to allow further cellular activity and remodeling. They could be stored in various liquids, dried or frozen. Also, the constructs could be produced by combining both living and decellularized thread of the same or differing cell types.

The cell synthesized thread can be used to produce these constructs using methods known in the art of textile production including but not limited to weaving, knitting, braiding, sewing, or molding. The construct, if made from a living thread can be, completely or partially, devitalized using methods described earlier for the living cell-synthesized thread. The construct can further be modified, completely or partially, by cross-linking as described earlier for the cell-synthesized thread. After or at any time during the construct production, new cells can be added to the construct. The cells can be of the same or of a different type than the ones used for the thread production, or a combination of cell populations. These cells can be non-autologous human cells (allogeneic), animal cells, genetically modified cells (human or animal) or any combination thereof. After, or at any time during in the production of the construct, the construct can be mechanically conditioned, completely or partially, in any or in multiple directions, by compressive, magnetic, isometric, isotonic, modulated or other types or regiments of forces including fluid shear stress. The construct can be stored in culture media under such conditions as to maintain cells alive and to allow the construct to be further remodeled by cell activity. The construct can also be stored in various liquids, dried or frozen. The living construct can be frozen using methods known in the art to maintain partial viability.

The construct can be produced by combining living and decellularized thread. The construct can be produced by combining one or more living or decellularized threads produced by different cell populations. Exogenous material, preferably in the form of filament can be woven, knitted or otherwise combined with living thread to form a hybrid construct. These exogenous materials could provide desirable mechanical properties such as elasticity and mechanical strength. They could also provide desirable biological activity by releasing biologically active compounds, including but not limited to antiproliferation agents and antithrombotic agents. These materials could later be removed to form gaps in the construct, such as channels, and allow for the production of more complex constructs and structures which could prove important for surgical devices used in drug delivery or wound healing. These materials could also serve other purposes including, but not limited to, facilitating identification, surgical use, healing, storage or further manipulation. These materials could be natural or synthetic and permanent or resorbable in nature. One example of these materials could be a biopsy taken from the patient, another human or an animal.

The exogenous materials could also include a “control rod” (as described in U.S. patent application Ser. No. 10/318,279) or another object that was present during the growth phase and embedded in the sheet used to produce the cell-synthesized thread, including magnetic and electronic objects such as radio frequency identification tags. Exogenous objects embedded in the tissue can also perform functional roles such as that of helping to connect the engineered tissue to the native tissue, e.g., suturing devices, cannulating devices, anchoring devices. Multiple constructs can be stacked or otherwise assembled, and further maintained in appropriate culture conditions. These construct assemblies can incorporate exogenous materials and objects as mentioned above for the case of a single construct. These construct assemblies can be modified as described above for the simple construct and the cell-synthesized thread including devitalization, cross-linking, and mechanical conditioning. New cells can be added to this construct assembly to create a more complex tissue or organ. The cells can be of the same or of a different type than the ones used for the thread production, or a combination of cell populations. These cells can be non-autologous human cells (allogeneic), animal cells, genetically modified cells (human or animal) or any combination thereof.

Threads made by methods and apparatus described herein, as well as art-recognized equivalents such as those after-arising, can be combined with tissue-engineered sheets, where, e.g., fine-structure is required or desired. Threads as described herein can be used to provide supporting structures for organ repair, as well as replacement of almost any bodily tissue, and used in applications including but not limited to, repair of blood vessels, hernias, and ligaments or tendons.

It is to be understood that although the methods and apparatus described herein are typically applied to create tissue threads for use in humans, the methods and apparatus could also be recruited to create tissue threads for veterinary and agricultural applications, such as for pets and farm animals. Typically the methods and apparatus herein will be applied to mammals, though such methods could also be used to create tissue for use in birds, reptiles, amphibians, and aquatic organisms such as fish.

The threads as described herein can be made in a number of dimensions. For example, where a ribbon is cut from a sheet of cells, the ribbon may have a width ranging from 100 microns to 20 cm. For example, such an exemplary upper limit can apply when making a ligament, for example, with one broad sheet twisted into one big thread. Other ranges of ribbon width include 1 mm to 10 cm, 0.5 cm to 5 cm, and 1-2 cm. Exemplary widths are 0.4, 0.6, and 0.8 cm.

Typically, when winding a ribbon into a thread, there is a 25-fold decrease in dimension, as has been seen with a 5 mm size ribbon. It would be understood that different factors will apply for other widths of ribbon.

A dry thread width made by the methods described herein may have a diameter from 5 microns to 1 cm, about 20 μM to about 1000 μM, or 100-800 μm, or 250 μM to about 500 μM, for example. Specific embodiments have diameters in the range 80-120 μm.

Lengths of threads made by methods and apparatus described herein can be between about 0.5 cm in fine-scale applications to up to 5 m in long applications. Threads as long as 3 m can be made by such methods. Other threads have lengths in the range 1 cm-1 m, or between 5 cm and 50 cm, or between 10 and 20 cm.

### EXAMPLES

### Example 1

### Autologous Suture Produced In Vitro

A cell-synthesized thread produced by methods described elsewhere herein can be used as a completely biological suture, cannulating device or anchoring device. It can be combined with suturing needles using various methods known in the art. The sutures can be produced in a wide variety of dimensions and having diameters commonly used in surgeries. If the patient's own cells are used, the suture can be completely autologous. Exogenous objects embedded in the tissue could also perform functional roles such as helping to connect the engineered tissue to the natural tissue. The use of living sutures can provide healing advantages due to the biological effects of the living cells. The completely biological nature of these sutures, either living or decellularized, may avoid any inflammatory reactions typically associated with foreign materials and could lead to a better healing. The sutures can be used dry so as to expand into the wound when they are rehydrated by fluid. This can be of particular interest in situations where bleeding through the needle holes can be a problem. Sutures can also be produced from non-autologous human cells (allogeneic), from animal cells, from genetically modified cells (human or animal) or any combination thereof.

### Example 2

### Woven Patches

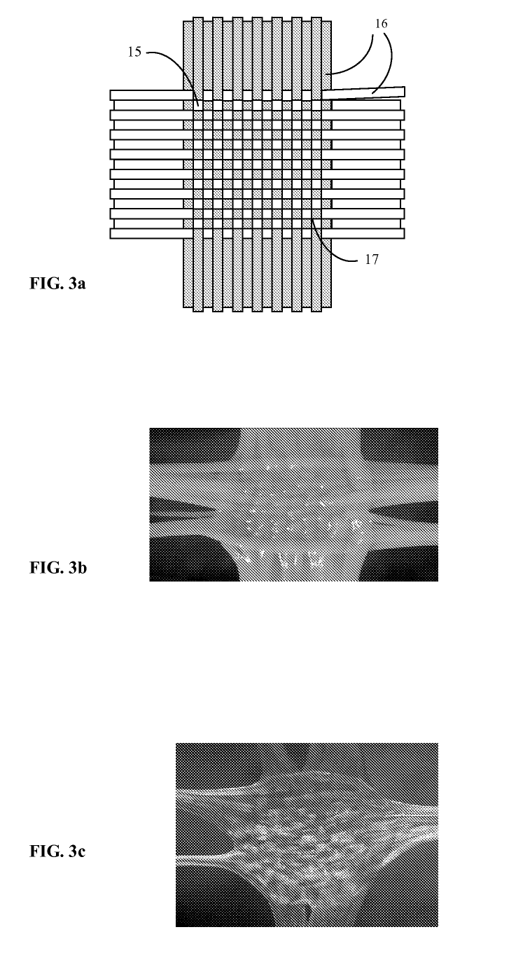

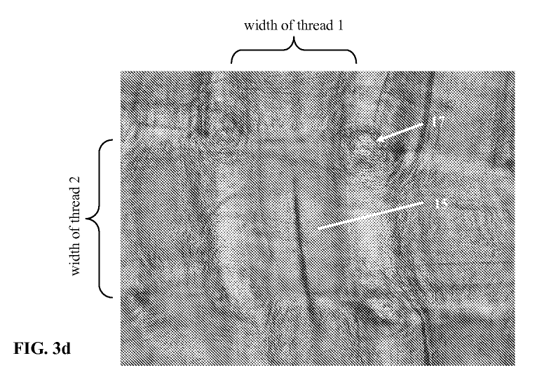

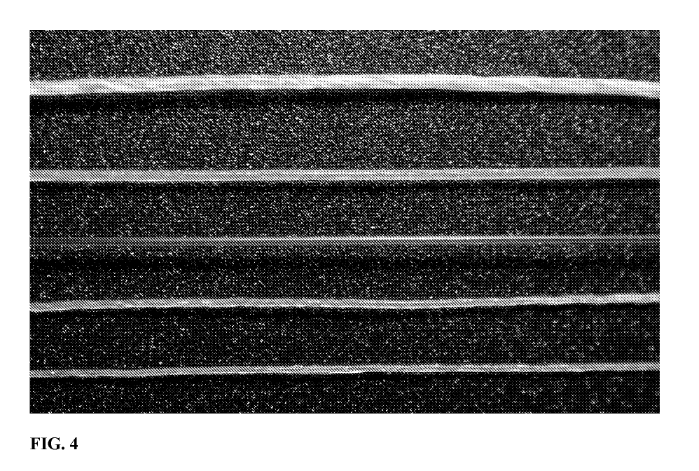

As described elsewhere herein, a living or decellularized cell-synthesized thread can also be used as a building block for the production of more complex structures. One example is the production of a planar patch. This can be achieved by methods such as, but not limited to, weaving or knitting the living or decellularized thread into a planar patch of any desired size, thickness and geometry. In a woven pattern for example, referring now to FIG. 3a, a planar patch is formed from eleven (11) threads by six (6) threads. At intersection 15 of living threads 16, the tissue's thickness is made of two layers of thread. At the four corners of intersection 15, channel-like structures are formed. When compared to the use of a simple sheet, this construct has the critical advantage of creating a very strong and cohesive patch by concentrating a large quantity of material into a small area.

Referring now to FIG. 3b, a micrograph depicting the intersection of threads in a dried woven patch is shown. The edges of both vertical and horizontal threads are seen as darker lines.

Intersections are also seen as small eddies where two threads overlap. FIG. 3b depicts the close proximity of overlapping threads that can be achieved through weaving. The living tissue shown in FIG. 3b, was produced by weaving 12 by 13 threads. It is approximately 6 mm wide and was produced from a sheet of approximately 90 mm wide before detachment. This provides, instantly, a 15-fold concentration of the tissue. Combined with the two-fold concentration due to the fact that this simple pattern creates two layers, a total of approximately 30-fold tissue concentration is achieved.

The tissue shown in FIG. 3c is a devitalized patch obtained after drying. A microscopic view of this tissue is shown in FIG. 3d, where the intersection 15 of two threads can be visualized as well as the accompanying channels 17. In this example, thread width varies between approximately 300 and 450 gm and channel diameter is about 70 μm. The channels are separated by 450 and 550 μm, though different geometries can be envisioned.

The patch could be used for applications such as, but not limited to, the production of a skin product for burn or ulcer treatment, the production of more complex tissues and organs (liver, pancreas, kidney), the production of a heart valve, the repair of myocardial defects or damage in pediatric and other patients, the repair of arterial walls, the repair of the meninges, the replacement or repair of ligament or tendons, and the repair of hernias.

### Example 3

### Tubular Constructs

In another embodiment of the present technology, the cell-synthesized threads can be assembled into a tubular structure, such as a tube, of any desired inner diameter, wall thickness and length.

Before, after or at any time during the tube production, new cells can be added to the tube. Cells can be seeded specifically on the inner surface of the tube. For the creation of a blood vessel, these cells could be endothelial, endothelial precursors, mesothelial, stem cells, etc. Before, after or at any time during the tube production, the tube can be mechanically conditioned, completely or partially, in any or in multiple directions, by compressive, magnetic, isometric, isotonic, modulated or other types or regiments of forces including fluid sheer stress. The tube can be stored in culture media under such conditions as to maintain cells alive and to allow cell proliferation and the tube to be further remodeled by cell activity. The tube can also be stored in various liquids, dried or frozen. The tube can be stored frozen using methods known in the art to maintain partial viability. Two or more tubes can be telescopically or otherwise assembled to create more complex structures, and further maintained in appropriate culture conditions. These tubes can be produced from the same or from different cell populations in order, for example, to reproduce the natural cellular organization of a blood vessel. New cells can be added to these tube assemblies to produce more complex constructs. In the particular case of a blood vessel equivalent, it may be interesting to produce a tube with flared ends to facilitate attachment to a blood vessel in vivo. In addition, exogenous materials that may be woven, knitted or otherwise combined with the cell-synthesized thread to form a hybrid construct could include anastomotic devices, staples, clips or other attachment devices to facilitate attachment to a blood vessel in vivo. As is often the case in vascular surgery, the resulting tube can be cut to size and at various angles. The cut ends can be treated to avoid fraying by methods including, but not limited to, crimping, gluing and stitching. The ends can also be everted and the anastomosis performed near but not at the ends.

Uses for such a living or decellularized tube include, but are not limited to, use as a vascular graft, as a vascular patch, as a guide for nerve regeneration, as a ureter or urethra replacement, as a bile duct replacement, as an intestine replacement, as a vas deferens replacement, or as a tendon or ligament replacement.

### Example 4

### Cavernous Structure

In a another embodiment of the technology, the cell-synthesized threads can be assembled into a cavernous shape with one or more chambers. For example, designs with single chambers can be adopted for use in the production of a bladder or lung. Holes can be incorporated into the tissue design to provide connectivity to liquid or gas conduits (e.g., bronchus, urethra, ureter). Specialized epithelial cells can be seeded in the cavities. Other cells can be seeded in the wall or the outer surface of the construct such as smooth muscle cells. Endothelial cells can be used to provide vascularity. For example, designs with four chambers can be used to mimic heart functionality. Alternatively or in addition, designs with multiple chambers could be used to produce networks of channels and bulky organs, including, but not limited to, liver, kidney and pancreas. Concentric shapes can be produced to allow complex wall organization. Specialized cells (hepatocytes, endothelial cells, epitheliums) can be seeded in independent channel systems to provide additional biological functions. All of the possible adaptations previously mentioned could also be used in conjunction with these more complex constructs.

### Example 5

### Applications of Patches

Patches, such as the one described in example 2, can be transformed into more complex constructs by using additional cell types and/or the inclusion of channels for fluid flow. Specialized cells such as pancreatic islet cells or hepatocytes could also be included to produce tissue engineered pancreas or liver. The channels would facilitate the distribution of blood or culture products.

The foregoing description is intended to illustrate various aspects of the present technology. It is not intended that the examples presented herein limit the scope of the present technology. The technology now being fully described, it will be apparent to one of ordinary skill in the art that many changes and modifications can be made thereto without departing from the spirit or scope of the appended claims.

What is claimed:

1. An tissue engineered biocompatible thread composition comprising one or more biological cells and an extracellular matrix synthesized by the cells.

2. The composition of claim 1, as isolated from a culture medium.

3. The composition of claim 1, wherein the biological cells are fibroblasts, endothelial cells, mesothelial cells, smooth muscle cells, cardiac or skeletal muscle cells, epithelial cells, urothelial cells, bone marrow cells, neural cells, glial cells, keratinocytes, fibrocytes, hepatocytes, chondrocytes, osteoblasts, angioblasts, myoblasts, adult stem cells, embryonic stem cells or a combination thereof.

4. The composition of claim 3, wherein the cells have been genetically engineered to express an exogenous nucleic acid.

5. The composition of claim 1, wherein the extracellular matrix comprises one or more naturally occurring extracellular matrix molecules or fragment thereof selected from: collagens, laminins, fibronectins, vitronectins, tenascins, integrins, elastins, fibrins, fibrilins, chitosans, celluloses, hyaluronic acids, chondroitin sulfates, dermatan sulfates, heparin sulfates, and keratin sulfates.

6. The composition of claim 5, wherein the exogenous nucleic acid encodes an extracellular matrix polypeptide.

7. The composition of claim 1, wherein diameter of the thread is from about 20 μM to about 1000 μM.

8. The composition of claim 1, further comprising a therapeutic agent.

9. The composition of claim 8, wherein the therapeutic agent is one or more a growth factor, a protein, an extracellular matrix component, a nucleic acid, a virus, an antibody, an antibiotic, an antiseptic, a chemotherapeutic agent, a small organic molecule, or a biological cell.

10. The composition of claim 9, wherein the growth factor is vascular endothelial cell growth factor, platelet derived growth factor, fibroblast growth factor, epidermal growth factor, hepatocyte growth factor, neurotrophic growth factor, insulin-derived growth factor, transforming growth factor, colony stimulating factors, tumor necrosis factor, an interleukin, an erythropoietin, or an interferon.

11. The composition of claim 9, wherein the extracellular matrix component is collagen, laminin, fibronectin, vitronectin, tenascin, integrin, elastin, fibrin, fibrilin, chitosan, cellulose, hyaluronic acid, chondroitin sulfate, dermatan sulfate, heparin sulfate, or keratin sulfate.

12. The composition of claim 1, wherein the cells have been devitalized.

13. The composition of claim 1, further comprising a natural or synthetic polymer.

14. A method of making a tissue engineered biocompatible thread composition, the method comprising:
(a) seeding a population of cells in a culture vessel; and
(b) culturing the cells under conditions that allow the formation of a tissue sheet comprised of cells and extracellular matrix synthesized by the cells in contact with the surface of the culture vessel; and
(c) cutting the sheet into one or more strips thereby forming a biocompatible thread composition.

15. The method of claim 14, wherein the cells comprise fibroblasts, endothelial cells, mesothelial cells, smooth muscle cells, cardiac or skeletal muscle cells, epithelial cells, urothelial cells, bone marrow cells, neural cells, glial cells, keratinocytes, fibrocytes, hepatocytes, chondrocytes, osteoblasts, angioblasts, myoblasts, adult stem cells, embryonic stem cells or a combination thereof.

16. The method of claim 15, wherein the cells have been genetically engineered to express an exogenous nucleic acid.

17. The method of claim 15, wherein the exogenous nucleic acid encodes an extracellular matrix polypeptide.

18. The method of claim 14, wherein the surface comprises a planar surface or a convex surface.

19. The method of claim 18, wherein the convex surface is disposed on a cylindrical structure.

20. The method of claim 14, wherein the cutting comprises a spiral or pattern that forms one contiguous thread, a bifurcated thread, or a network of connected threads.

21. The method of claim 24, wherein the cutting comprises forming a spiral or pattern that forms a contiguous thread.

22. The method of claim 14, further comprising detaching the thread from the substrate.

23. The method of claim 14, removing the tissue sheet from the substrate before the cutting.

24. The method of claim 14, wherein the width of the strips is from about 0.1 cm to about 1 cm.

25. The method of claim 14, further comprising twisting the strips to form a thread.

26. The method of claim 14, further comprising substantially decellularizing the tissue sheet or the biocompatible thread composition.

27. The method of claim 14, further comprising providing a therapeutic agent and (a) culturing the adherent cells in the presence the therapeutic agent or (b) associating the therapeutic agent with the tissue sheet or the formed biocompatible thread composition.

28. The method of claim 27, wherein associating the therapeutic agent with the tissue sheet or the formed biocompatible thread composition comprises covalently bonding the therapeutic agent to the tissue sheet or the formed biocompatible thread composition.

29. The method of claim 28, wherein the therapeutic agent is one or more of a growth factor, a protein, an extracellular matrix component, a nucleic acid, a virus, an antibody, an antibiotic, an antiseptic, a chemotherapeutic agent, a small organic molecule, or a biological cell.

30. The method of claim 29, wherein the growth factor is a vascular endothelial cell growth factor, platelet derived growth factor, fibroblast growth factor, epidermal growth factor, hepatocyte growth factor, neurotrophic growth factor, insulin-derived growth factor, transforming growth factor, colony stimulating factors, tumor necrosis factor, an interleukin, an erythropoietin, or an interferon.

31. The method of claim 29, wherein the extracellular matrix component is collagen, laminin, fibronectin, vitronectin, tenascin, integrin, elastin, fibrin, fibrilin, chitosan, cellulose, hyaluronic acid, chondroitin sulfate, dermatan sulfate, heparin sulfate, or keratin sulfate.

32. The method of claim 14, further comprising treating the tissue sheet or the biocompatible thread composition with a cross-linking agent.

33. A method of making a tissue engineered biocompatible thread composition, the method comprising:
(a) seeding a population of adherent cells on the surface of a cylinder contained within a culture vessel, wherein the surface of the cylinder comprises a plurality of circumferential grooves; and
(b) culturing the cells under conditions that allow the formation of a tissue sheet comprised of cells and extracellular matrix synthesized by the cells in contact the grooves; and
(c) removing the tissue sheet from the grooves, thereby forming a biocompatible thread composition.

34. The method of claim 33, wherein the grooves are from about 10 μm to about 1 mm in width.

35. The method of claim 33, wherein the sheet is cut longitudinally into one or more threads.

36. A tissue engineered biocompatible thread composition made by the method of claim 14 or claim 33.

37. A tissue engineering construct comprising the biocompatible thread composition of any of claim 1, 14 or 38.

38. The tissue engineering construct of claim 37, wherein the threads are twisted, braided or bundled to form a filament, or woven, interlaced or tied to form a mesh.

39. Use of the biocompatible thread composition of any of claim 1, 14 or 38 in the preparation of a medicament.

40. A method of treatment comprising:
(a) identifying a mammalian subject as having a recipient organ or tissue in need of repair; and
(b) placing the biocompatible thread composition of any of claim 1, 14 or 33 or the tissue engineering construct of claim 42 in or on the organ or tissue.

41. The method of claim 45 wherein the subject is human.

42. An article of manufacture comprising: a measured amount of a tissue engineered biocompatible thread composition and one or more of packaging material, or a package insert comprising instructions for use, and a sterile container.

43. A bioreactor for use in growing tissue comprising:
a cylindrical support having an external surface capable of supporting cellular growth;
wherein the cylindrical support is enclosed in a chamber configured to provide conditions for cellular growth.

44. The bioreactor of claim 43 further comprising at least one control rod.

45. The bioreactor of claim 43, wherein the cylinder is at least partially immersed in a liquid solution of standing or circulating media capable of supporting or enhancing tissue growth.

46. The bioreactor of claim 49, wherein the external surface of the cylinder is grooved or patterned.
